(19) United States
(12) Patent Application Publication
(10) Pub. No.: US 2025/0288010 A1
(43) Pub. Date: Sep. 18, 2025

(54) ATOMIZER AND ELECTRONIC ATOMIZATION DEVICE

(71) Applicant: Shenzhen Verdewell Technology Limited, Shenzhen (CN)

(72) Inventors: Xiaojun ZHONG, Shenzhen (CN); Shuai LUO, Shenzhen (CN); Diansheng BAI, Shenzhen (CN); Yaqiao WANG, Shenzhen (CN); Gama YANG, Shenzhen (CN); Ju XIE, Shenzhen (CN)

(21) Appl. No.: 19/074,814

(22) Filed: Mar. 10, 2025

(30) Foreign Application Priority Data
Mar. 18, 2024 (CN) 202410322873.8

(51) Int. Cl. A24F 40/48; A24F 40/10; A24F 40/42; A24F 40/44; A24F 40/46; A24F 40/485; A61M 11/04

(52) U.S. Cl. CPC A24F 40/48; A24F 40/10; A24F 40/42; A24F 40/44; A24F 40/46; A24F 40/485; A61M 11/042

(57) ABSTRACT

An atomizer includes: a housing assembly, a liquid storage cavity and an air guiding channel being formed in the housing assembly; and an atomization core mounted in the housing assembly in a longitudinal direction of the atomizer. The liquid storage cavity and the atomization core are arranged in the longitudinal direction of the atomizer. A liquid guiding channel is arranged in a center of the atomization core, one end of the liquid guiding channel being in communication with the liquid storage cavity, an other end of the liquid guiding channel being closed. An outer peripheral wall of the atomization core is in communication with the air guiding channel.

### CROSS-REFERENCE TO PRIOR APPLICATION

Priority is claimed to Chinese Patent Application No. 202410322873.8, filed on Mar. 18, 2024, the entire disclosure of which is hereby incorporated by reference herein.

### FIELD

This application relates to the field of atomization technologies, and more specifically, to an atomizer and an electronic atomization device.

### BACKGROUND

In general, an electronic atomization device includes an atomizer and a power supply component. The power supply component is configured to supply power to the atomizer. The atomizer includes a liquid storage cavity and an atomization core. The liquid storage cavity is configured to store a liquid medium, and the atomization core is configured to heat and atomize the liquid medium after being powered on, to generate aerosols that can be absorbed.

In an existing atomizer, two ends of the atomization core are respectively in communication with the liquid storage cavity, so that the liquid mediums on the two ends of the atomization core are not consumed equally, and e-liquid shortage is likely to occur at one end during use. In order to connect the two ends of the atomization core to ensure that e-liquid infiltrates the atomization core at all times, a connection channel needs to be formed outside the atomization core to connect the two ends of the atomization core. Consequently, in this solution, the atomizer needs to be formed relatively thick, and therefore, the atomizer is seriously limited in styling.

### SUMMARY

In an embodiment, the present invention provides an atomizer, comprising: a housing assembly, a liquid storage cavity and an air guiding channel being formed in the housing assembly; and an atomization core mounted in the housing assembly in a longitudinal direction of the atomizer, wherein the liquid storage cavity and the atomization core are arranged in the longitudinal direction of the atomizer, wherein a liquid guiding channel is arranged in a center of the atomization core, one end of the liquid guiding channel being in communication with the liquid storage cavity, an other end of the liquid guiding channel being closed, and wherein an outer peripheral wall of the atomization core is in communication with the air guiding channel.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a three-dimensional schematic diagram of an atomizer according to an embodiment of this application;

FIG. 2 is a schematic longitudinal cross-sectional view of an atomizer parallel to a center of an atomization core according to an embodiment of this application;

FIG. 3 is a schematic longitudinal cross-sectional view of a housing assembly of an atomizer parallel to a center of an air inlet channel according to an embodiment of this application;

FIG. 4 is a schematic structural diagram of an atomization assembly in an atomizer according to an embodiment of this application;

FIG. 5 is a schematic structural diagram of an atomization assembly in an atomizer with one base body being removed according to an embodiment of this application;

FIG. 6 is a schematic structural diagram of a base body in an atomizer according to an embodiment of this application;

FIG. 7 is a schematic longitudinal cross-sectional view of a main housing in an atomizer according to an embodiment of this application;

FIG. 8 is a cross-sectional view of an atomization core in an atomizer according to an embodiment of this application; and

FIG. 9 is a schematic structural diagram of an atomizer with a main housing being removed according to an embodiment of this application.

### DETAILED DESCRIPTION

In an embodiment, the present invention provides an atomizer and an electronic atomization device, to resolve a technical problem existing in the prior art that two ends of an atomization core are respectively in communication with a liquid storage cavity, causing e-liquid shortage at one end.

In an embodiment, the present invention provides an atomizer, including a housing assembly and an atomization core. A liquid storage cavity and an air guiding channel are formed in the housing assembly. The atomization core is mounted in the housing assembly in the longitudinal direction of the atomizer, and the liquid storage cavity and the atomization core are arranged in the longitudinal direction of the atomizer. A liquid guiding channel is arranged in the center of the atomization core, one end of the liquid guiding channel is in communication with the liquid storage cavity, and the other end of the liquid guiding channel is closed. The outer peripheral wall of the atomization core is in communication with the air guiding channel.

In an embodiment, the air guiding channel and the liquid storage cavity are spaced along the transverse direction of the housing assembly.

In an embodiment, the housing assembly further forms an air inlet channel in communication with the outer peripheral wall of the atomization core, and the air inlet channel and the air guiding channel are respectively arranged on the two transverse sides of the atomization core.

In an embodiment, the atomizer further includes an atomization base, the atomization base is mounted in the housing assembly, the atomization base forms an atomization cavity, and the atomization core is mounted in the atomization cavity; and the air inlet channel and the air guiding channel are respectively in communication with the atomization cavity.

In an embodiment, a first cavity and a second cavity are formed in the housing assembly, the first cavity and the second cavity are respectively arranged on the two opposite sides of the atomization base, and the first cavity and the second cavity are respectively in communication with the atomization cavity; the air inlet channel is connected with the first cavity, and the air inlet of the air inlet channel is higher than the bottom surface of the first cavity; and the second cavity is connected with the air guiding channel.

In an embodiment, the cross-sectional area of the air guiding channel is less than the cross-sectional area of the second cavity, and a first step is formed at a junction of the air guiding channel and the second cavity.

In an embodiment, a liquid guiding hole is connected between the liquid storage cavity and the liquid guiding channel, and at least one capillary channel is formed on the inner wall of the liquid guiding hole.

In an embodiment, the liquid guiding hole is formed on the atomization base, a connecting hole communicating the liquid guiding hole with the liquid storage cavity is formed on the housing assembly, the connecting hole and the liquid guiding hole are arranged in a stair shape, and the inner diameter of the connecting hole is less than the inner diameter of the liquid guiding hole.

In an embodiment, the atomizer further forms a vent channel in communication with the liquid storage cavity.

The vent channel is formed on the housing assembly;
the vent channel is formed between the atomization base and the housing assembly; or
the vent channel is formed between the atomization base and the atomization core.

In an embodiment, the atomization core includes a supporting member, a liquid guiding assembly sleeved outside the supporting member, and a heating wire wound outside the liquid guiding assembly. The liquid guiding channel is formed in the supporting member, and a liquid guiding port is formed on the side wall of the supporting member.

In an embodiment, the housing assembly includes a main housing and a base.

The liquid storage cavity and the air guiding channel are separately formed in the main housing, and the air inlet channel is formed in the base.

The main housing further forms a cavity with the top respectively in communication with the liquid storage cavity and the air guiding channel, and with an opening at the bottom.

The base covers the bottom opening of the cavity, and the base and the main housing are enclosed to form the first cavity, the second cavity, and a mounting cavity for mounting the atomization base.

According to another aspect, this application further provides an electronic atomization device, including a power supply component and the foregoing atomizer.

A beneficial effect of the atomizer and the electronic atomization device provided in this application is that: the atomization core is mounted in the housing assembly in the longitudinal direction of the atomizer, one end of the liquid guiding channel in the center of the atomization core is in communication with the liquid storage cavity, and the other end of the liquid guiding channel is closed. Therefore, only one end of the atomization core is in communication with the liquid storage cavity, thereby avoiding unequal consumption of liquid mediums at the two ends of the atomization core, avoiding a case of e-liquid shortage at one end during use, and further reducing the thickness of the atomizer, and styling of the atomizer is not limited. In addition, in this application, the liquid storage cavity and the atomization core are arranged along the longitudinal direction of the atomizer. Therefore, when a user uses the atomizer, the atomization core in the liquid storage cavity can flow to the liquid guiding channel of the atomization core under an action of gravity of the atomization core, thereby improving flowability of the liquid mediums, and ensuring smooth liquid flowing.

### Reference Numerals in the Accompanying Drawings

100. Housing assembly; 110. main housing; 111. liquid storage cavity; 112. air guiding channel; 113. connecting hole; 114. second step; 115. cavity; 116. first limiting groove; 117. vent channel; 120. base; 121. first airway; 122. sealing pillar; 130. sealing base; 131. second airway; 132. second limiting groove; 140. sealing cap; 150. air inlet channel; 151. air inlet; 160. first cavity; 170. second cavity; 171. first step; 180. mounting cavity; 200. atomization core; 210. supporting member; 211. liquid guiding channel; 212. liquid guiding port; 220. liquid guiding assembly; 221. heat conduction layer; 222. isolation layer; 230. heating wire; 300. atomization base; 310. base body; 320. atomization cavity; 330. mounting hole; 340. liquid guiding hole; 341. capillary channel; 350. limiting block; and 400. electrode.

To make technical problems to be resolved by this application, technical solutions, and beneficial effects clearer, the following further describes this application in detail with reference to the accompanying drawings and embodiments. It should be understood that specific embodiments described herein are only used for explaining this application, and are not used for limiting this application.

It should be noted that, when a component is referred to as “being fixed to” or “being arranged on” another component, the component may be directly on the another component or indirectly on the another component. When an element is referred to be “connected to” another element, the element may be directly connected to the another component, or indirectly connected to the another component.

It should be understood that, orientation or position relationships indicated by terms such as “length”, “width”, “upper”, “lower”, “front”, “rear”, “left”, “right”, “vertical”, “horizontal”, “top”, “bottom”, “inner”, and “outer” are orientation or position relationships shown based on the accompanying drawings, and are merely used for describing this application and simplifying the description, rather than indicating or implying that the mentioned apparatus or element should have a particular orientation or be constructed and operated in a particular orientation, and therefore, should not be construed as a limitation to this application.

In addition, the terms “first” and “second” are used for descriptive purposes only and are not to be construed as indicating or implying relative importance or implicitly indicating the number of technical features indicated. Therefore, a feature restricted by “first” or “second” may explicitly indicate or implicitly include one or more of such features. In the descriptions of this application, “a plurality of” means two or more, unless it is specifically defined otherwise.

In an existing atomizer, an atomization core of which is arranged transversely, two ends of the atomization core are respectively in communication with a liquid storage cavity, so that liquid mediums on the two ends of the atomization core are not consumed equally, and e-liquid shortage is likely to occur at one end during use. In order to connect the two ends of the atomization core to ensure that e-liquid infiltrates the atomization core at all times, a connection channel needs to be formed outside the atomization core to connect the two ends of the atomization core. Consequently, in this design, the atomizer needs to be formed relatively thick, and therefore, the atomizer is seriously limited in styling.

To resolve the foregoing problems, embodiments of this application provide an atomizer and an electronic atomization device. An atomization core 200 is mounted in a housing assembly 100 in the longitudinal direction of the atomizer (which is referred to as the longitudinal direction for short below for ease of description), one end of the atomization core 200 in the longitudinal direction is connected to a liquid storage cavity 111, and the other end of the atomization core 200 in the longitudinal direction is closed, that is, only one end of the atomization core 200 is connected to the liquid storage cavity 111. In this way, unequal consumption of liquid mediums at the two ends of the atomization core 200 can be avoided, and e-liquid shortage at one end during use is avoided, thereby reducing the thickness of the atomizer, and styling of the atomizer is not limited.

The following describes the electronic atomization device provided in the embodiments of this application. The electronic atomization device includes an atomizer and a power supply component. The power supply component usually includes a battery configured to supply power to the atomizer and a control circuit configured to control heating of the atomizer. The atomizer is configured to accommodate a liquid medium, and heat and atomize the liquid medium after power-on to generate aerosols. The liquid medium includes, but is not limited to, materials used for medical, regimen, health, and cosmetic purposes.

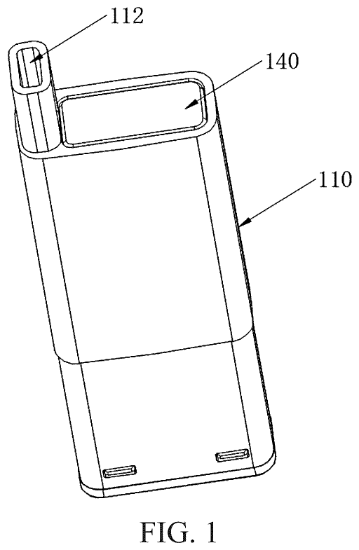

Referring to FIG. 1 to FIG. 3, the atomizer provided in the embodiments of this application is now described. The atomizer includes a housing assembly 100 and an atomization core 200. A liquid storage cavity 111 and an air guiding channel 112 are formed in the housing assembly 100. The atomization core 200 is mounted in the housing assembly 100 in the longitudinal direction of the atomizer, the liquid storage cavity 111 and the atomization core 200 are arranged in the longitudinal direction of the atomizer. A liquid guiding channel 211 is arranged at the center of the atomization core 200, one end of the liquid guiding channel 211 is in communication with the liquid storage cavity 111, the other end of the liquid guiding channel 211 is closed, and the outer peripheral wall of the atomization core 200 is in communication with the air guiding channel 112.

The longitudinal direction of the atomizer refers to a direction parallel or substantially parallel to the height direction of the atomizer when the atomizer is placed vertically. The transverse direction of the atomizer and the longitudinal direction of the atomizer are perpendicular to each other. The transverse direction of the atomizer refers to the horizontal direction of the atomizer or a direction substantially parallel to the horizontal direction of the atomizer when the atomizer is placed vertically.

The liquid storage cavity 111 is configured to store the liquid medium, the center of the atomization core 200 forms a liquid guiding channel 211, the inner peripheral wall of the atomization core 200 is a liquid inlet surface, and the outer peripheral wall of the atomization core 200 is an atomization surface. The liquid medium in the liquid storage cavity 111 enters the liquid guiding channel 211 through an end of the liquid guiding channel 211, is absorbed by the side wall of the atomization core 200, and is atomized on the outer peripheral wall of the atomization core 200 to form aerosols. The outer peripheral wall of the atomization core 200 is in communication with the air guiding channel 112, so that the aerosols formed through atomization of the atomization core 200 can be guided out by the air guiding channel 112 for an user to inhale.

In the atomizer provided in the embodiments of this application, the atomization core 200 is mounted in the housing assembly 100 in the longitudinal direction of the atomizer, one end of the liquid guiding channel 211 in the center of the atomization core 200 is in communication with the liquid storage cavity 111, and the other end of the liquid guiding channel 211 is closed. Therefore, only one end of the atomization core 200 is in communication with the liquid storage cavity 111, thereby avoiding unequal consumption of liquid mediums at the two ends of the atomization core 200, avoiding e-liquid shortage at one end during use, and further reducing the thickness of the atomizer, and styling of the atomizer is not limited. In addition, in this application, the liquid storage cavity 111 and the atomization core 200 are arranged along the longitudinal direction of the atomizer. Therefore, when a user uses the atomizer, the atomization core 200 in the liquid storage cavity 111 can flow to the liquid guiding channel 211 of the atomization core 200 under actions of gravity of the atomization core 200 and negative pressure suction, thereby improving flowability of the liquid medium, and ensuring smooth liquid flowing. In addition, an existing limitation that liquid flowing needs to be performed on guiding channels on both sides of the liquid storage cavity 111 is further eliminated, making the entire atomizer lighter and thinner.

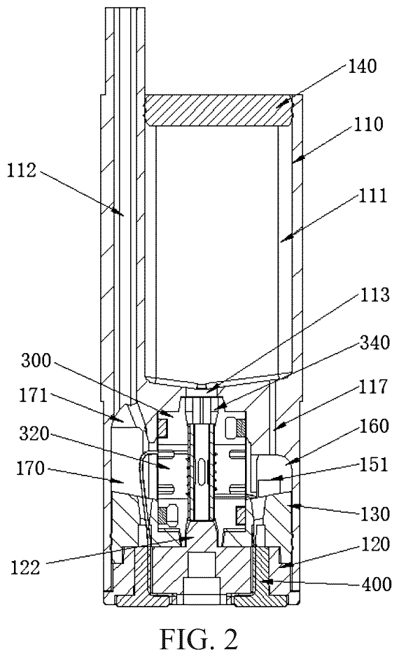

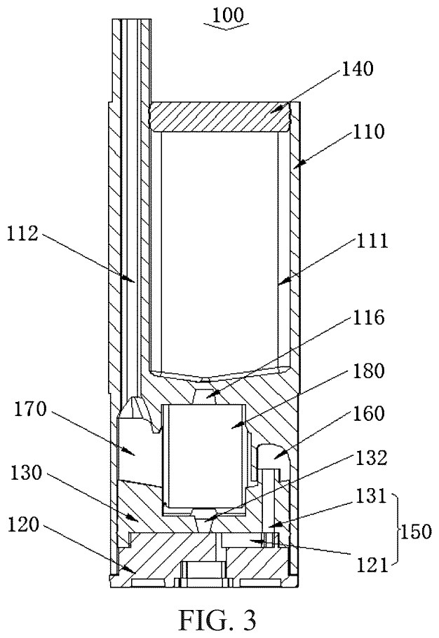

In an embodiment, referring to FIG. 2 and FIG. 3, the air guiding channel 112 and the liquid storage cavity 111 are spaced apart in the transverse direction of the housing assembly 100, the liquid storage cavity 111 is in communication with the inner peripheral wall (that is, a liquid guiding surface) of the atomization core 200, and the air guiding channel 112 is in communication with the outer peripheral wall (that is, the atomization surface) of the atomization core 200. In this application, the liquid storage cavity 111 and the air guiding channel 112 are spaced apart, to be specific, the air guiding channel 112 is arranged outside the liquid storage cavity 111. Therefore, the liquid storage cavity 111 can directly communicate with the atomization core 200 vertically, instead of communicating with the atomization core 200 through the periphery of the air guiding channel 112, thereby simplifying a communication structure between the liquid storage cavity 111 and the atomization core 200. In addition, the air guiding channel 112 does not need to run through the liquid storage cavity 111, and the air guiding channel 112 does not occupy space of the liquid storage cavity 111. Therefore, the liquid storage cavity 111 is more spacious, and the flowability of the liquid medium in the liquid storage cavity 111 is improved. In addition, structures of the air guiding channel 112 and the liquid storage cavity 111 are simplified, and the atomization core 200 is placed more easily.

In an embodiment, referring to FIG. 2 and FIG. 3, the housing assembly 100 further forms an air inlet channel 150 in communication with the outer peripheral wall of the atomization core 200, and the air inlet channel 150 and the air guiding channel 112 are respectively arranged on two transverse sides of the atomization core 200. When the atomization core 200 is powered on to atomize the liquid medium to form the aerosols, an airflow entering through the air inlet channel 150 is transversely around the peripheral of the atomization core 200 to carry the aerosols to the air guiding channel 112, and finally, the airflow carrying the aerosols is guided out through the air guiding channel 112 for the user to inhale. In this embodiment, the air inlet channel 150 and the air guiding channel 112 are respectively arranged on two transverse sides of the atomization core 200, and in this way, the airflow can transversely flow through the atomization core 200 to carry the aerosols out.

In an embodiment, referring to FIG. 2 and FIG. 3, the air guiding channel 112, the atomization core 200, and the air inlet channel 150 are sequentially arranged along the longitudinal direction of the atomizer, and in this way, the airflow can flow from bottom to top, thereby reducing airflow flow resistance.

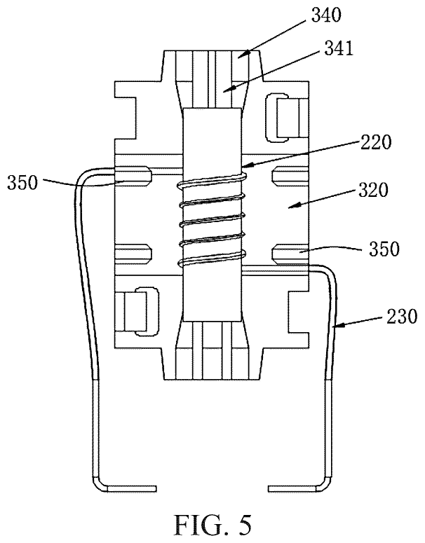

In an embodiment, referring to FIG. 2 and FIG. 5, the atomizer further includes an atomization base 300. The atomization base 300 is mounted in the housing assembly 100, the atomization base 300 is enclosed to form an atomization cavity 320, and the atomization core 200 is mounted in the atomization cavity 320. The air inlet channel 150 and the air guiding channel 112 are respectively in communication with the atomization cavity 320.

The atomization core 200 is in communication with the liquid storage cavity 111 through the atomization base 300, and the liquid medium in the liquid storage cavity 111 flows into the liquid guiding channel 211 of the atomization core 200. When the atomization core 200 is powered on, the outer peripheral wall of the atomization core 200 starts to heat and atomize the liquid medium, and the outer peripheral wall of the atomization core 200 is located in the atomization cavity 320. Therefore, the aerosols formed through atomization in the atomization core 200 are accommodated in the atomization cavity 320, carried to the air guiding channel 112 by an airflow flowing from the air inlet channel 150 into the atomization cavity 320, and guided out for the user to inhale.

In an embodiment, referring to FIG. 2 and FIG. 3, a first cavity 160 and a second cavity 170 are formed on the housing assembly 100. The first cavity 160 and the second cavity 170 are respectively arranged on two opposite sides of the atomization base 300, and the first cavity 160 and the second cavity 170 are respectively in communication with the atomization cavity 320. The air inlet channel 150 is in communication with the first cavity 160, an air inlet 151 of the air inlet channel 150 is higher than the bottom surface of the first cavity 160, and the second cavity 170 is in communication with the air guiding channel 112. In this embodiment, because the air inlet 151 of the air inlet channel 150 is higher than the bottom surface of the first cavity 160, space in the first cavity 160 between the air inlet 151 and the bottom surface of the first cavity 160 may be configured to collect e-liquid. Similarly, space in the second cavity 170 between the air inlet 151 and the bottom surface of the second cavity 170 may be configured to collect e-liquid. For case of description, the space in the first cavity 160 and the space in the second cavity 170 that may be configured to collect e-liquid are referred to as e-liquid collection space. The e-liquid collection space may be configured to collect condensate generated at the atomization core 200 or a liquid medium slightly leaked during use, and the collected liquid contacts with the atomization core 200, and may be consumed during inhalation. In addition, the air inlet 151 is higher than the e-liquid collection space to avoid blockage at the atomization core 200.

In an embodiment, referring to FIG. 2 and FIG. 3, the bottom surface of the first cavity 160 extends obliquely downward in a direction toward the atomization core 200 from the side facing away from the atomization core 200, and the bottom surface of the second cavity 170 extends obliquely downward in a direction toward the atomization core 200 from the side facing away from the atomization core 200. In this way, the condensate and the leaked liquid medium can be collected toward the atomization core 200 as much as possible, so that the condensate and the leaked liquid can be consumed during inhalation.

In an embodiment, referring to FIG. 2, the cross-sectional area of the air guiding channel 112 is less than the cross-sectional area of the second cavity 170, and a first step 171 is formed at the junction between the air guiding channel 112 and the second cavity 170. In this way, when the atomizer is laid flat, liquid collected in the e-liquid collection space is blocked by the first step 171, thereby preventing the liquid from flowing into the air guiding channel 112 or even flowing out of the air guiding channel 112 when the atomizer is laid flat. In an embodiment, referring to FIG. 2, the cross-sectional area of the inner peripheral wall of the first step 171 gradually decreases from the second cavity 170 to the air guiding channel 112. In this way, an airflow in the second cavity 170 can be guided to the air guiding channel 112, and condensation caused by the airflow remaining at an edge of a side of the first step 171 facing the second cavity 170 is avoided, and structural stress concentration is reduced.

In an embodiment, a mounting cavity 180 is further formed in the housing assembly 100. The mounting cavity 180 is in communication between the first cavity 160 and the second cavity 170, the atomization base 300 is mounted in the mounting cavity 180 and is located below the liquid storage cavity 111. The first cavity 160 is in communication with the air inlet channel 150, and the second cavity 170 is in communication with the air guiding channel 112. The air inlet channel 150 is in communication with the atomization core 200 through the first cavity 160, and the air guiding channel 112 is in communication with the atomization core 200 through the second cavity 170.

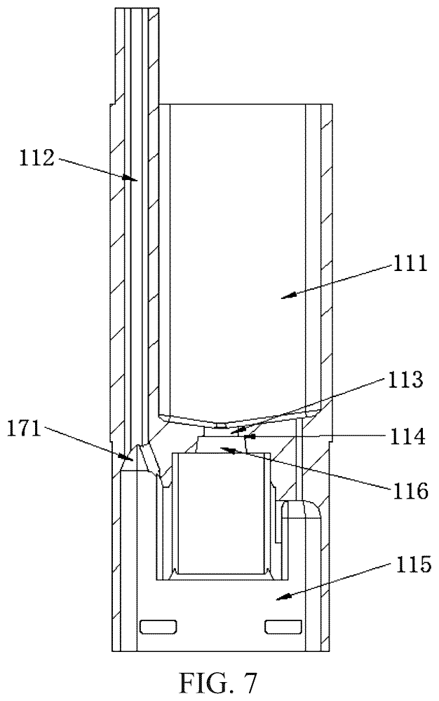

In an embodiment, referring to FIG. 2, FIG. 5, and FIG. 7, a liquid guiding hole 340 is connected between the liquid storage cavity 111 and the liquid guiding channel 211, and at least one capillary channel 341 is formed on the inner wall of the liquid guiding hole 340. The capillary channel 341 may adsorb and collect a part of the liquid medium. When the atomizer is placed horizontally, the liquid medium in the liquid guiding channel 211 may not completely flow to the liquid storage cavity 111, but a part is collected in the capillary channel 341. Therefore, when a user places the atomizer vertically and inhales, the liquid medium in the capillary channel 341 can quickly flow to the liquid guiding channel 211, thereby atomizing the liquid medium, avoiding a case in which the liquid medium in the liquid guiding channel 211 completely leaves the atomization core 200 when the atomizer is placed flat, and avoiding inhaling a scorched liquid medium during use by the user because when the atomizer is placed upright, the liquid medium cannot quickly flow back into the liquid guiding channel 211 due to the high viscosity of the liquid medium.

In an embodiment, a plurality of capillary channels 341 exist, and the plurality of capillary channels 341 are sequentially arranged at intervals in the circumferential direction of the liquid guiding hole 340. In this way, the liquid medium can be stored in the plurality of capillary channels. Therefore, when the atomizer is placed upright, sufficient liquid medium can quickly flow to the liquid guiding channel 211.

In an embodiment, referring to FIG. 2 and FIG. 5 to FIG. 7, the liquid guiding hole 340 is formed on the atomization base 300, a connecting hole 113 that connects the liquid guiding hole 340 with the liquid storage cavity 111 is formed on the housing assembly 100. The connecting hole 113 and the liquid guiding hole 340 are arranged in a stair shape, and the inner diameter of the connecting hole 113 is less than the inner diameter of the liquid guiding hole 340.

In this embodiment, the connecting hole 113 and the liquid guiding hole 340 are arranged in a stair shape, that is, a second step 114 is formed at a joint between the connecting hole 113 and the liquid guiding hole 340, and the liquid medium in the liquid guiding hole 340 is blocked from flowing to the connecting hole 113 through the second step 114, thereby avoiding that the liquid medium in the liquid guiding channel 211 completely leaves the atomization core 200 when the atomizer is placed flat, and avoiding that during use by the user, due to high viscosity of the liquid medium, the liquid medium cannot quickly flow back into the liquid guiding channel 211 when being placed upright and a scorched liquid medium is inhaled.

In an embodiment, referring to FIG. 2, a vent channel 117 is further formed in the atomizer, and the vent channel 117 is in communication with the liquid storage cavity 111. The vent channel 117 is arranged in a way that when air pressure in the liquid storage cavity 111 is low, the liquid storage cavity 111 can be in communication with an external atmosphere through the vent channel 117, thereby reducing situations of leaks during inhaling and placing. In addition, the arrangement of the vent channel 117 further improves the flowability of the liquid medium in the liquid storage cavity 111, so that the liquid medium can smoothly flow to the atomization core 200.

In an embodiment, referring to FIG. 2, one end of the vent channel 117 is in communication with the liquid storage cavity 111, the other end of the vent channel 117 is in communication with the atomization cavity 320, and the atomization cavity 320 may communicate with the external atmosphere through the air inlet channel 150, thereby implementing communication between the liquid storage cavity 111 and the external atmosphere. It may be understood that, in other embodiments of this application, one end of the vent channel 117 may be in communication with the liquid storage cavity 111, and the other end may be directly in communication with the external atmosphere. This is not uniquely limited herein.

In an embodiment, referring to FIG. 2, the vent channel 117 is formed on the housing assembly 100. In other words, through forming the vent channel 117 on the housing assembly 100, one end of the vent channel 117 is in communication with the liquid storage cavity 111, and the other end of the vent channel 117 is in communication with the atomization cavity 320 or directly with the external atmosphere.

In a specific embodiment, referring to FIG. 2, the vent channel 117 is formed on the housing assembly 100, and the vent channel 117 extends in the longitudinal direction of the atomizer. The top end of the vent channel 117 is in communication with the liquid storage cavity 111, the bottom end of the vent channel 117 is in communication with the first cavity 160, and the vent channel 117 sequentially passes through the first cavity 160 and the air inlet channel 150 to communicate with the external atmosphere. It may be understood that, in other embodiments of this application, the vent channel 117 may alternatively be bent and extend, or may be formed through multiple times of bending, and an end of the vent channel 117 facing away from the liquid storage cavity 111 may alternatively extend to the second cavity 170, or directly extend to the outer side of the housing assembly 100. This is not uniquely limited herein.

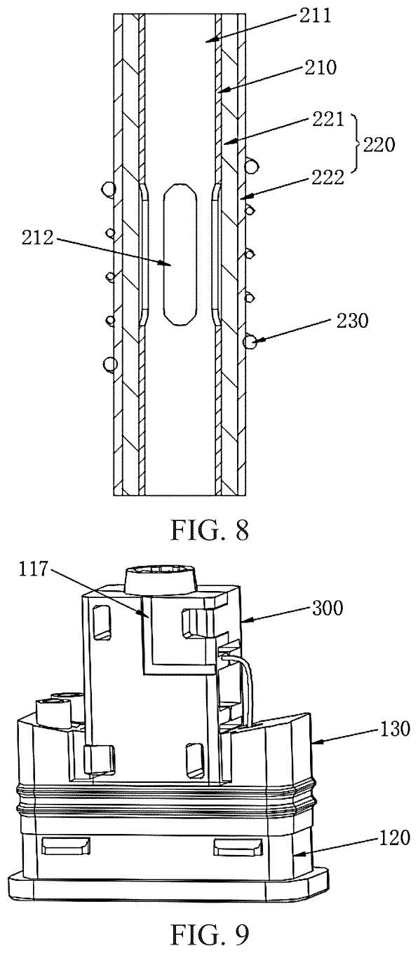

In another embodiment of this application, the vent channel 117 is formed between the atomization base 300 and the housing assembly 100. For example, referring to FIG. 8, the vent channel 117 is formed on a side of the atomization base 300 facing toward the housing assembly 100; or the vent channel 117 is formed on a side of the housing assembly 100 facing toward the atomization base 300; or the vent channel 117 is respectively formed on a side of the atomization base 300 facing toward the housing assembly 100 and a side of the housing assembly 100 facing toward the atomization base 300.

Referring to FIG. 8, the vent channel 117 is formed on a side of the atomization base 300 facing toward the housing assembly 100, the vent channel 117 is L-shaped, one end of the vent channel 117 is in communication with the liquid storage cavity 111, and the other end of the vent channel 117 is in communication with the atomization cavity 320. It may be understood that, in other embodiments of this application, the vent channel 117 may alternatively be provided in a multi-bend structure, and the other end of the vent channel 117 passes through the housing assembly 100 to communicate with the external atmosphere.

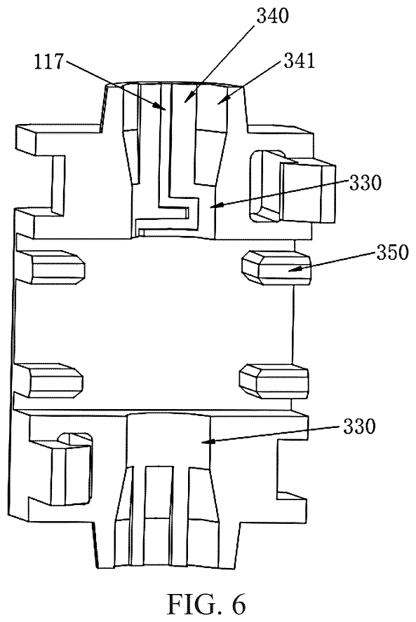

In another embodiment of this application, referring to FIG. 6, the vent channel 117 is formed between the atomization base 300 and the atomization core 200. Specifically, the vent channel 117 is formed on a side of the atomization base 300 facing toward the atomization core 200, one end of the vent channel 117 is in communication with the liquid storage cavity 111, and the other end of the vent channel 117 is in communication with the atomization cavity 320. The vent channel 117 may be linear, or may be formed by bending and connecting multiple linear channels.

In an embodiment, referring to FIG. 5 and FIG. 6, two mounting holes 330 respectively arranged on two opposite sides of the atomization cavity 320 in the longitudinal direction of the atomizer are formed on the atomization base 300, the two mounting holes 330 are coaxially arranged, and two opposite ends of the atomization core 200 respectively penetrate the two mounting holes 330.

Specifically, the mounting hole 330 located at the top end is in communication with the liquid guiding hole 340.

In an embodiment, referring to FIG. 2, a sealing pillar 122 extends from the housing assembly 100 to the mounting hole 330 at the bottom, and the sealing pillar 122 seals the bottom end of the mounting hole 330. The bottom end of the atomization core 200 is inserted into the mounting hole 330 and abuts against the sealing pillar 122. In this way, the bottom end of the atomization core 200 can be sealed by the sealing pillar 122, and the atomization core 200 can be longitudinally limited by the sealing pillar 122.

In an embodiment, referring to FIG. 8, the atomization core 200 includes a supporting member 210, a liquid guiding assembly 220 sleeved outside the supporting member 210, and a heating wire 230 wound outside the liquid guiding assembly 220. A liquid guiding channel 211 is formed in the supporting member 210, and a liquid guiding port 212 is formed on the side wall of the supporting member 210. A liquid medium in the liquid storage cavity 111 flows to the liquid guiding channel 211, flows to the liquid guiding assembly 220 through the liquid guiding port 212, and is stored in the liquid guiding assembly 220. When the heating wire 230 is powered on and performs heating, the heating wire 230 atomizes the liquid medium the an outer peripheral wall of the liquid guiding assembly 220 to form aerosols. In this embodiment, the liquid medium is guided into the atomization core 200, and the heating wire 230 is wound around the outer peripheral wall of the atomization core 200, so that the outer peripheral wall of the atomization core 200 is an atomization surface of the atomization core 200, thereby increasing an atomization surface area of the atomization core 200, and improving atomization efficiency.

In an embodiment, referring to FIG. 8, the liquid guiding assembly 220 includes a heat conduction layer 221 and an isolation layer 222, the heat conduction layer 221 is sleeved outside the supporting member 210, the isolation layer 222 is sleeved outside the heat conduction layer 221, and the heating wire 230 is wound outside the isolation layer 222. The heat conduction layer 221 is configured to conduct the liquid medium, and is further configured to conduct heat of the heating wire 230 to the supporting member 210, thereby reducing viscosity of the liquid medium near the heat conduction layer 221, and improving the flowability. The isolation layer 222 is configured to isolate the heat conduction layer 221 and the heating wire 230, to prevent the two from being electrically conductive.

In an embodiment, referring to FIG. 2, two electrodes 400 are mounted on the housing assembly 100. The two electrodes 400 are respectively fixedly connected to two ends of the heating wire 230, and the two electrodes 400 are respectively electrically connected to the power supply component, to supply power to the heating wire 230 by using the power supply component.

In an embodiment, referring to FIG. 1 and FIG. 2, the housing assembly 100 includes a main housing 110 and a base 120. The liquid storage cavity 111 and the air guiding channel 112 are respectively formed in the main housing 110, and the air inlet channel 150 is formed on the base 120. The main housing 110 further forms a cavity 115 with the top portion in communication with the liquid storage cavity 111 and the air guiding channel 112 respectively, and with an opening at the bottom. The base 120 covers the bottom opening of the cavity 115, and the base 120 and the main housing 110 are enclosed to form the first cavity 160, the second cavity 170, and a mounting cavity 180 for mounting the atomization base 300.

The housing assembly 100 is divided into two parts: the main housing 110 and the base 120, thereby facilitating formation of the liquid storage cavity 111, the air guiding channel 112, the first cavity 160, the second cavity 170, the mounting cavity 180, and the air inlet channel 150, and facilitating assembly of the atomization base 300 and the atomization core 200.

In an embodiment, referring to FIG. 2 and FIG. 3, the housing assembly 100 further includes a sealing base 130. The sealing base 130 is mounted on a side of the base 120 facing toward the liquid storage cavity 111. The outer peripheral wall of the sealing base 130 elastically seals and abuts against the inner peripheral wall of the main housing 110. The sealing base 130, the base 120, and the main housing 110 are jointly enclosed to form the first cavity 160, the second cavity 170, and the mounting cavity 180. The sealing pillar 122 is formed on the base 120. The scaling base 130 is arranged to seal the bottom opening of the cavity 115, thereby ensuring that the liquid medium in the first cavity 160 or the second cavity 170 leaks outward. In addition, because the atomization base 300 is mounted between the sealing base 130 and the main housing 110, the sealing base 130 is further arranged to enhance sealing between the atomization base 300 and the main housing 110, that is, increase a sealing cavity of the liquid storage cavity 111.

In an embodiment, referring to FIG. 3, the air inlet channel 150 is arranged sequentially running through the base 120 and the sealing base 130. Specifically, a first airway 121 is formed on the base 120, a second airway 131 is formed on the sealing base 130. One end of the first airway 121 is in communication with the external atmosphere, one end of the second airway 131 is in communication with the other end of the first airway 121, and the other end of the second airway 131 extends above the top side of the sealing base 130 to extend into the first cavity 160 for a specific height.

Referring to FIG. 2 and FIG. 3, because two electrodes 400 need to be mounted on the base 120, the electrodes 400 have a large size, and the sealing pillar 122 needs to extend into the atomization core 200 from the center of the base 120, the first airway 121 is bent and extends on the base 120. Actually, regardless of a shape design of the first airway 121, it is only necessary that the second airway 131 is in communication with the external atmosphere.

In an embodiment, referring to FIG. 2, a top opening of the liquid storage cavity 111 is formed, a sealing cap 140 is mounted at the top opening of the liquid storage cavity 111, and the scaling cap 140 is configured to seal the top opening of the liquid storage cavity 111. The arrangement of the top opening facilitates injection of the liquid medium into the liquid storage cavity 111, and the arrangement of the sealing cap 140 can enable the top of the liquid storage cavity 111 to be in a sealed state when the atomizer is used or carried, to avoid e-liquid leakage at the top.

In an embodiment, referring to FIG. 3, a first limiting groove 116 is formed at a position on the main housing 110 corresponding to the atomization base 300, a second limiting groove 132 is formed at a position on the sealing base 130 corresponding to the atomization base 300. The first limiting groove 116 and the second limiting groove 132 are respectively configured to limit two opposite ends of the atomization base 300, thereby ensuring that the atomization base 300 is securely mounted in the housing assembly 100.

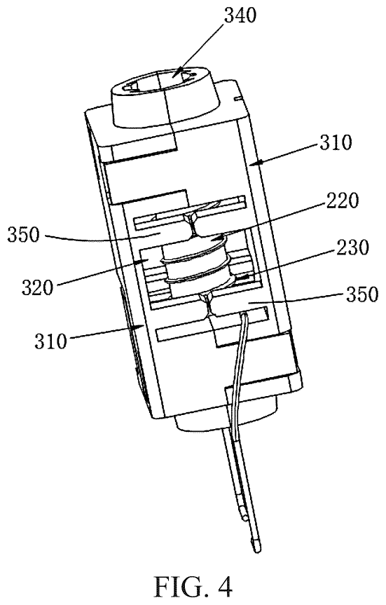

In an embodiment, referring to FIG. 4, the atomization base 300 includes two base bodies 310, the two base bodies 310 are oppositely buckled to form the atomization base 300, and the two base bodies 310 are jointly enclosed to form the foregoing atomization cavity 320.

In an embodiment, referring to FIG. 4 to FIG. 6, a limiting block 350 is formed on the atomization base 300 on two opposite sides of the atomization core 200. The limiting block 350 is configured to limit and guide the heating wire 230 led out of the atomization core 200, to form a longitudinal limit of the atomization core 200 on the atomization base 300.

Specifically, referring to FIG. 4 to FIG. 6, the atomization core 200 generally leads out two ends of the heating wire 230, and the two ends respectively extend out along the first cavity 160 and the second cavity 170 to be respectively connected to the two electrodes 400. To ensure limits of the two ends, the atomization base 300 is provided with at least two limiting blocks 350. In addition, to ensure that the heating wire 230 can be limited when being mounted on the atomization base 300 in a forward direction and a backward direction, two limiting blocks 350 are arranged on the atomization base 300 on the two opposite sides of the atomization core 200, to limit the ends of the heating wire 230.

When the atomizer of this application is assembled, the atomization core 200 may be first assembled, then the atomization core 200 is assembled on one base body 310, and a longitudinal direction of the atomization core 200 is limited by the limiting block 350 on the base body 310. Then, the other base body 310 is placed to cover to complete assembly of a heating assembly. Then, the sealing base 130 is sleeved on the base 120, and the heating assembly is inserted into the sealing base 130. Finally, the base 120, the sealing base 130, and the heating assembly are integrally mounted into the main housing 110. This modular assembly is simple.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. An atomizer, comprising:
a housing assembly, a liquid storage cavity and an air guiding channel being formed in the housing assembly; and
an atomization core mounted in the housing assembly in a longitudinal direction of the atomizer,
wherein the liquid storage cavity and the atomization core are arranged in the longitudinal direction of the atomizer,
wherein a liquid guiding channel is arranged in a center of the atomization core, one end of the liquid guiding channel being in communication with the liquid storage cavity, an other end of the liquid guiding channel being closed, and
wherein an outer peripheral wall of the atomization core is in communication with the air guiding channel.

2. The atomizer of claim 1, wherein the air guiding channel and the liquid storage cavity are spaced along a transverse direction of the housing assembly.

3. The atomizer of claim 1, wherein the housing assembly forms an air inlet channel in communication with the outer peripheral wall of the atomization core, and
wherein the air inlet channel and the air guiding channel are respectively arranged on two transverse sides of the atomization core.

4. The atomizer of claim 3, further comprising:
an atomization base mounted in the housing assembly, the atomization base forming an atomization cavity, the atomization core being mounted in the atomization cavity,
wherein the air inlet channel and the air guiding channel are respectively in communication with the atomization cavity.

5. The atomizer of claim 4, wherein a first cavity and a second cavity are formed in the housing assembly, the first cavity and the second cavity being respectively arranged on two opposite sides of the atomization base, the first cavity and the second cavity being respectively in communication with the atomization cavity,
wherein the air inlet channel is connected with the first cavity, and an air inlet of the air inlet channel is higher than a bottom surface of the first cavity, and
wherein the second cavity is connected with the air guiding channel.

6. The atomizer of claim 5, wherein a cross-sectional area of the air guiding channel is less than a cross-sectional area of the second cavity, and
wherein a first step is formed at a junction of the air guiding channel and the second cavity.

7. The atomizer of claim 4, wherein a liquid guiding hole is connected between the liquid storage cavity and the liquid guiding channel, and
wherein at least one capillary channel is formed on an inner wall of the liquid guiding hole.

8. The atomizer of claim 7, wherein the liquid guiding hole is formed on the atomization base,
wherein a connecting hole communicating the liquid guiding hole with the liquid storage cavity is formed on the housing assembly,
wherein the connecting hole and the liquid guiding hole are arranged in a stair shape, and
wherein an inner diameter of the connecting hole is less than an inner diameter of the liquid guiding hole.

9. The atomizer of claim 4, further comprising a vent channel in communication with the liquid storage cavity,
wherein the vent channel is formed on the housing assembly, the vent channel is formed between the atomization base and the housing assembly, or the vent channel is formed between the atomization base and the atomization core.

10. The atomizer of claim 1, wherein the atomization core comprises a supporting member,
wherein a liquid guiding assembly sleeved outside the supporting member,
wherein a heating wire is wound outside the liquid guiding assembly,
wherein the liquid guiding channel is formed in the supporting member, and
wherein a liquid guiding port is formed on a side wall of the supporting member.

11. An electronic atomization device, comprising:
a power supply component; and
the atomizer of claim 1.
